SEQUENCE LISTING (3 sequences)

SEQ ID NO: 1 - DNA, 1729 bp, terrestrial pit viper Bothrops jararaca
  CDS (211)...(1008)
  ref <301>: AUTHORS: Camargo, A.C.M.; Portaro, F.C.V.; Murbach, A.F.; Ianzer, D.; Farsky, S.H.P.; Palma, M.S.; and Hayashi, M.A.F.
  ref <302>: TITLE: The C-type natriuretic peptide precursor of snake brain contain highly specific inhibitors of the angiotensin-converting enzyme.
  ref <303>: JOURNAL: Journal of Neurochemistry
  ref <304>: VOLUME: submitted
  ref <308>: DATABASE ACCESSION NUMBER: GenBank AF171670
  ref <309>: DATABASE ENTRY DATE: 2001-10-15
gaattcggcacgaggaaagcgatgccgacgctgcgagcgaatctaggccaagcagacaagcgcggcttgacgagacgctcttgcggcaaagcgcccggctcgactcggctcccttcggctcagcggtccgcgccgtcgaggattctcactcttgctcgctcgctcgctcgctctcgcccaccctctctgccggcgcctgcgcagcccgggatggtcctctcccgcctggcggccagcgggctgctgctcctggccctgctggcgctctcggtcgacgggaagccggtgcagcagtgggcgcaagggggctggccgcgccccggtcctgagattccgccgctgaaggtgcagcagtgggcgcaagggggctggccgcgccccggtcctgagattccgccgctgacagtgcagcagtgggcgcaaaactggccgcatcctcagattccgccgctgacggtgcagcagtgggcgcaatgggggcggccgccgggtcctccgattccgccgctgacggtgcagcagtgggcgcaagcgcggccgccgcatcctccgataccgccggcgccgctgcagaagtgggcgccggtgcagaagtgggcgccgctgctgcagccccacgagagtccggcgagcggcacgacggccttgcgggaggagctgagcctggggccagaagcggcgtcgggcgtcccgtctgctggagcagaggtcgggcgcagcggctcgaaggcgcccgctgcaccccataggctgtcgaagagcaaaggggcggcggcgacctcggcggcgtcgcggccgatgcgggacttgcgccccgacggcaagcaggcgcggcaaaactggggccggatggtgcaccacgaccaccacgcagcagtaggaggcggcggcggcggcggaggaggaggagcgcgtcgtctgaaggggctggccaagaaaggggcggccaagggctgcttcggcctgaaggtcgaccgcatcggcaccatgagtggcctgggctgctgaagccgtcgagggcggcgcgaaggcgacacctggcggcggtctttgaactgagaccccaccccatccgcggacatttctggacatcccctgcaattcatccagggatcccaggcccacacagcctgtctcctgttggtacgagcaacttgaagaagccatttttcagtgggagggttctgggtagggttggactgagttttgcgcccccaaatcttcctatcttacctaatatatacctagatgccatatatatatcgatagatacaagtacacccaacacaaaaaacacgcccgcccacaacttcttccccgcgacaaaggacggtgtcgggagacggagagaaccagctgcgtatgccatcgagattgctgtttgtacattcgtgtcacgcataaatgtatttaagttgtaaaagctatttatatattgtttataaagagatatttataaaaaaaaattttatttatgtaaactaaacgaaaaggggtcgagcattgtaaactttttttttttccccctttctcatcctgaatagtcggaaggaaagaaaaaaaaaatcatggacgatttgataacctgtcttgcattttaatcggatgcagtctttctttccgaaatgggtggtttgaacaaggcttttagataccacggataagagtaaaaaatgcatttgtaaagaaatcgcacgctatctaccaaaggtgtaaaagaacctgacgc

SEQ ID NO: 2 - protein, 265 aa, terrestrial pit viper Bothrops jararaca
  ref <308>: DATABASE ACCESSION NUMBER: GenBank AAD51326
  ref <309>: DATABASE ENTRY DATE: 2001-10-15
MVLSRLAASGLLLLALLALSVDGKPVQQWAQGGWPRPGPEIPPLKVQQWAQGGWPRPGPEIPPLTVQQWAQNWPHPQIPPLTVQQWAQWGRPPGPPIPPLTVQQWAQARPPHPPIPPAPLQKWAPVQKWAPLLQPHESPASGTTALREELSLGPEAASGVPSAGAEVGRSGSKAPAAPHRLSKSKGAAATSAASRPMRDLRPDGKQARQNWGRMVHHDHHAAVGGGGGGGGGGARRLKGLAKKGAAKGCFGLKVDRIGTMSGLGC

SEQ ID NO: 3 - protein, 4 aa, Unknown
  Oligopeptide substrate used in the Enzymatic assay of Example 7
ASDK

(19) United States
(12) Patent Application Publication
(10) Pub. No.: US 2005/0031604 A1
(43) Pub. Date: Feb. 10, 2005

(54) Isolation and purification procedure of vasopeptidase peptide inhibitors

(76) Inventors: Antonio Carlos Martins de Camargo, Sao Paul (BR); Fernanda Calheta Vieira Portaro, Sao Paulo (BR); Alessandra Ferragini Murbach, Sao Paulo (BR); Danielle Alvez Ianzer, Sao Paulo (BR); Sandra Helena Poliselli Farsky, Sao Paulo (BR); Mario Sergio Palma, Sao Paulo (BR); Mirian Akemi Furuie, Sao Paulo (BR)

Correspondence Address: BIRCH STEWART KOLASCH & BIRCH, PO BOX 747, FALLS CHURCH, VA, 22040-0747, US

(21) Appl. No.: 10/471,931

(22) PCT Filed: Mar. 18, 2002
(86) PCT No.: PCT/BR02/00041

(51) Int. Cl. A61K 38/43; C12P 21/06; C12N 9/99

(52) U.S. Cl. 424/94.1; 435/68.1; 435/184

(57) ABSTRACT

The present invention patent refers to the isolation and purification of peptides secreted by serpent venom glands, specifically Bothrops jararaca; to the peptide thus obtained, as well as to the production procedures by genetic engineering techniques in procaryotic and eukaryotic systems; to the engineered peptide thus obtained; to the production of said peptide by chemical synthesis, as well as to the peptide resulting from this chemical processing. It also refers to the utilization of said peptides, obtained by different procedures, in distinct pharmaceutical compositions, and introduced into the organism by a variety of means, in order for them to act as inhibitors of vasopeptidases, and consequently reduce systemic arterial blood pressure, and show local vasodilating action.

The invention refers to the isolation and purification procedures of vasopeptidase peptide inhibitors, specific for the carboxylic site of the angiotensin converting enzyme, secreted by serpent venom glands (BPPs), or endogenously produced (EVASINS), presenting anti-hypertensive and vasodilator actions. It also refers to the isolation and determination procedures of the amino acid sequence of the peptide inhibitors secreted by serpent venom glands (BPPs), or endogenously produced (EVASINS). It also refers to the amino acid sequence determination procedure of BPPs by deduction of the cDNA of the precursors of these molecules, expressed in serpent tissues, particularly Bothrops jararaca; to the amplification procedure of the cDNA from brain or pancreas cDNA libraries, particularly from Bothrops jararaca; to the solid phase synthesis prodedure of vasopeptidase peptide inhibitors; to the use of vasopeptidase peptide inhibitors with vasodilator and anti-hypertensive action. It further refers to the determination procedure of the inhibitory activity on vasopeptidases, and of the biological activities on smooth muscle, and on the microcirculatory and the cardiovascular systems.

Specifically, the invention refers to the isolation of the peptides secreted by serpent venom glands (BPPs), or found endogenously, particularly in the brain of Bothrops jararaca. We suggest the generic name EVASINS (endogenous vasopeptidases inhibitors) for the endogenous peptides; to the peptides thus obtained, as well as to the procedure of producing them by genetic engineering methods in prokaryote and eukaryote systems; to the engineered peptides thus obtained; to the production of said peptides by chemical synthesis as well as to the peptides resulting from the proteolytic processing. It also refers to the use of the peptides obtained by the referred procedures in pharmaceutical compounds with inhibitory action on vasopeptidases, and consequently lowering blood pressure, and the use of the referred peptides to lower blood pressure.

Thus the invention refers to peptides with cardiovascular action, known as bradykinin potentiating peptides, secreted by serpent venom glands (BPPs), or produced in other tissues of Bothrops jararaca (EVASINS).

The serpent venom is composed of a complex mixture of biologically active proteins and peptides. Among known, characterized biologically active peptides, we list bradykinin potentiating peptides (BPPs), natriuretic factors (C-type natriuretic peptides, CNP), saraphotoxins (SRTx), among others. These bioactive peptides (hormones, neuropeptides, toxins, etc.) are generated by the hydrolytic action of enzymes on the proteic precursors of these peptides, which contain one or more bioactive peptides per molecule. Toxins secreted by the venom glands of various serpents, such as Bothorps jararaca, contain a large number of peptides with hypotensive action (BPPs), whose mechanism of action is related to its inhibitory effect on the angiotensin-converting enzyme (ACE), blocking the conversion of angiotensin I into angiotensin II (hypertensive), and preserving bradykinin (hypotensive).

The primary structure of these peptides allows their classification into two families: those peptides smaller than 7 amino acid residues, and those larger than 7 amino acid residues, showing a high degree of homology among them.

Research in this specific field showed that a large variety of applications can be found for toxins, particularly the venom toxins of serpents, or the modifications of some of these molecules, such as the bradykinin potentiating peptides (BPPs), which can be specific agents of anti-hypertensive action in mammals, including man.

The amino acid sequence of other bradykinin potentiating peptides, having high degree of homology with the amino acid sequence of BPPs, were not obtained from the venom of snakes. In fact, these were deducted from the nucleotide sequences of their precursors found in other serpent tissues, such as the brain, pancreas and spleen tissue, using molecular biology techniques. These endogenous peptides, in their synthetic form, like the BPPs secreted from the venom gland of the Bothrops jararaca, acting on the angiotensin converting enzyme (ACE) of the vascular endothelium, preventing the formation of angiotensin II (hypertensive) and preserving the hypotensive action of bradykinin.

The angiotensin converting enzyme (ACE) is a peptidase with two catalytic sites (carboxy- or C-site, and amino- or N-site), which are mainly located to the cytoplasmic membrane of endothelial cells. The C-terminal active site is more specific for angiotensin I and bradykinin. The BPPs act by inhibiting the ACE and, up to this invention, the specificity of the inhibition of the C- or N-sites was unknown, and the result of this inhibition is the reduction of arterial blood pressure.

Inhibition of the ACE, not selective for a specific active sites, led to the development of the site-directed inhibitor called captopril.

The N site metabolizes the natural circulating peptide hormone (Ac-Ser-Asp-Lys-Pro), which regulates hematopoiesis. The carboxylic domain of the enzyme (C site) is more specific for convertion of angiotensin I into angiotensin II and inactivates bradykinin. Up to this moment, it was not known whether all the described BPPs presented any preference for the C site, turning them into more specific antihypertensive agents.

There still are a number of unknown toxins from the the venoms of snakes, which may have the potential to act as anti-hypertensive agents. These molecules may not only inhibit specifically the C-site of the ACE, but even, and simultaneously, inhibit other peptidases of the vascular endothelium, as the neutral endopeptidase (NEP) and the endothelin converting enzyme (ECE). The ECE inactivates bradykinin, natriuretic peptides, hypotensive agents, and generates endothelins (hypertensive). The ACE, NEP and ECE are known as vasopeptidases, and are responsible for most of the arterial blood pressure control. These double and/or triple inhibitors are not only more specific, but also more efficient because they act on the metabolism of endogenous molecules regulating the arterial blood pressure. The distinction between the two catalytic sites of the ACE has been well-defined in: Dive, V.; Cotton J.; Yiotakis, M. A.; Vassiliou, S.; Jiracek, J.; Vazeaux, G.; Chauvet, G.; Cuniasse, P.; Corvol; RXP 407, a phosphinic peptide, is a potent inhibitor of angiotensin I converting enzyme able to differentiate between its two active sites. Proc. Natl. Acad. Sci. USA, 13, 4330-4335, (1999).

It is known from the scientific literature that some peptides from the venom of Brazilian and Asian serpents, presenting hypotensive action, have already been isolated and sequenced (Ondetti & Cushman, Ann.Rev.Biochem. 51, 293-308, 1982), and that the antihyperstensive efficiency of one of them has been demonstrated in humans (Gravas et al. N. Engl. J. Med. 291, 817-821, 1974).

Several BPPs have been deduced from the amino acid sequences of their precursors using molecular biology techniques (Murayama et. al., Proc. Natl. Acad. Sci. USA, 94, 1189-1193, 1997).

The pharmaceutical industry developed a new antihypertensive drug of non-peptidic nature and without any chemical analogies with endogenous molecules. It was called OMOPATRILAT, displaying inhibition properties for both the ACE and the NEP, presenting long-term anti-hypertensive efficiency in rats with high concentration of circulating renin. Besides, this substance improves cardiac performance, prolonging the survival of spontenously hypertensive rats, SUR, a model for human essential hypertension. The OMAPATRILAT is the first site-directed inhibitor for two vasopeptidases, the ACE and NEP, in advanced phase of clinical trials.

The major differences between the OMAPATRILAT and the BPPs and EVASINS are:

1—the BPPs and EVASINS are of polypeptidic nature;

2—they are synthetic molecules identical or homolog to peptides secreted by exocrine glands, or endogenously produced in vertebrates;

3—as ACE inhibitors, the BPs and EVASINS of 8 to 13 amino acid residues show preference for the C-site, and also inhibit the NEP X;

4—they are degraded mainly by humoral and tissue proteolytic enzymes;

The U.S. Pat. No. 5,538,9991, published Jul. 23^{rd}, 1996, and the U.S. Pat. No. 5,559,135, published Sep. 24^{th}, 1996, describe the inhibitor secreted by serpent venom glands—PCA-W-P-R-P-E-I-P-P-SQ 20881—an ACE inhibitor, which consequently reduces the arterial blood pressure in animals, and which showed efficient reduction of arterial blood pressure in hypertensive individuals. However, these patents do no describe or reveal the specificity of action of this inhibitor on one or the two catalytic sites of the ACE. The patents do not show either the importance of other enzymes capable of regulating the arterial blood pressure, therefore no research was done on BPPs with higher specificity of action.

The present invention differs from the aforementioned patents by demonstrating specificity of action of the EVASINS on vasopeptidases; it also establishes a clear relationship between the chemical structures of several dozens of these molecules with their activity on the cardiovascular system. This invention also establishes one structural motif and the endogenous origin of the leader molecules proposed here, features not predicted in the references quoted above.

The same arguments mentioned above can be used do differentiate the present invention from the USPTO patents U.S. Pat. No. 3,819,831, published Jun. 25^{th}, 1974, U.S. Pat. No. 3,714,140, published Jan. 30^{th}, 1973, U.S. Pat. No. 3,832,337, published Aug. 27^{th}, 1974, U.S. Pat. No. 3,849,252, published Nov. 19^{th}, 1974, U.S. Pat. No. 3,947,575, published Mar. 30^{th}, 1976, U.S. Pat. No. 3,973,006, published Aug. 3^{rd}, 1976, U.S. Pat. No. 4,731,439, published Mar. 15^{th}, 1988, U.S. Pat. No. 4,774,318, published Sep. 27^{th}, 1988, and U.S. Pat. No. 5,550,127, published Aug. 27^{th}, 1996.

Aiming at solving the existing technical problems, in order to obtain a final product of superior quality in terms of efficiency as anti-hypertensive agents, the present invention proposes, for the first time and in a novel manner, BPPs and EVASINS, presenting: (1) a defined structural motif; (2) specificity for the C-site of the ACE, and (3) high inhibitory specificity for the NEP X.

These novel specific anti-hypertensive agents for the C site of the ACE, presenting inhibitory action on vasopeptidase were isolated and sequenced from the venom of the Brazilian serpent Bothrops jararaca, or identified by cloning and cDNA sequencing, obtained from the tissues of this serpent. These peptides, which can also be obtained by chemical synthesis, and which contain the same amino acid sequence as the natural peptides, show long-term anti-hypertensive action in rats. The use of these synthetic peptides, which have the same sequence as the natural peptides, used as such, or chemically modified in order to maintain the characteristics described above, can be useful as therapeutic agents in the treatment of cardiovascular disorders or in local circulatory actions, particularly in hypertensive disorders in humans and other cardiovascular disorders.

Twenty-two (22) BPPs found in the venom and in tissues of Bothrops jararaca, were sequenced by mass spectrometry, or their sequences were deduced from cDNA sequences of the precursors of those molecules expressed in the tissues of the serpent. The corresponding synthetic peptides were tested as inhibitors of the C-site of the recombinant ACE and of NEP, and also both as potentiating agents of the bradykinin contractile activity on isolated guinea pig ileum, and as bradykinin hypotensive activity in rats. They were found both as BPPs (secreted) and as EVASINS (endogenous). They can be divided in two groups according to the specificity of action on the C site of the ACE:

a) non-specific. One peptide, the pentapeptide <E K W A P (Pyroglutamyl(<E)-Lysine(K)-tryptophane(W)-Alanine(A)-Proline (P), which, although not presenting a defined selectivity for the C-site of the ACE, presents high potentiating activity of bradykinin, as observed on the preparation of isolated smooth muscle as well as by its hypotensive effect. It was found as BPP (secreted from the serpent venom glands) as well as EVASINS (endogenous, from the serpent brain tissue). It presents Ki values in the μM range, for both the N and C sites of the ACE. It also inhibits, with low affinity, the NEP with Ki values ranging from 50-150 μM. It potentiates the contractile action of bradykinin on isolated guinea pig ileum with concentrations that vary from 15-300 nM. The hypotensive effect of bradykinin was also potentiated by 60% in concentrations of 300 nM, showing a 5-fold (five) increase in the duration of the hypotensive effect, when compared to the duration of the effect of bradykinin alone.

b) specific. These peptides present molecular masses ranging from 1000 to 1700 Daltons, containing 8 to 13 amino acid residues. In this group, the most selective and effective potentiators of the contractile action of the bradykinin on isolated guinea pig ileum and on rat arterial blood pressure, were found as BPPs or EVASINS. They were chemically modified generating other peptides with similar pharmacological properties.

The table 1 below presents the synthetic oligonucleotides derived from the inhibitors described in group b) for this present invention:

TABLE 1

| Formu- | |
|---|---|
| la | Sequences |
| I | pp¹aa¹aa²aa³P⁴aa⁵aa⁶P⁷P⁸ |
| II | pp¹aa¹aa²aa³aa⁴P⁵aa⁶aa⁷P⁸P⁹ |
| III | pp¹aa¹aa²aa³aa⁴aa⁵P⁶aa⁷aa⁸P⁹P¹⁰ |
| IV | pp¹aa¹aa²aa³aa⁴aa⁵aa⁶P⁷aa⁸aa⁹P¹⁰P¹¹ |
| V | pp¹aa¹aa²aa³aa⁴aa⁵aa⁶aa⁷P⁸aa⁹aa¹⁰P¹¹P¹² |
| VI | pp¹aa¹aa²aa³aa⁴aa⁵aa⁶aa⁷aa⁸P⁹aa¹⁰aa¹¹P¹²P¹³ |

Where:
P is always proline. All others are always L-amino acids and are presented in the one-letter-code (see below).
pp¹ is the N-terminal and can be also pyroglutamyl (<E) or any other simple amino acid, generally non-basic.
aa¹ is a non-basic amino acid, generally W, S, G or N;
aa² is a non-acid amino acid, generally P, G, W or R;
aa³ is a non-acid amino acid, generally P, A, R or W;
aa⁴ is P for formula I and it is generally the amino acid T, P, G, H, R, W or E for all other oligopeptides;
aa⁵ is generally Q, N, P, or G for formula I;
aa⁶ for formula II, aa⁷ for formula III, aa⁸ for formula IV and aa⁹ for formula V are non-basic amino acids, generally Q, N, P, or G.

aa⁶ for formula I, aa⁷ for formula II, aa⁸ for formula III, aa⁹ for formula IV and aa¹⁰ for formula V are always I, A or T.

| Acid amino acids - D, E | |
|---|---|
| Basic amino acids - K, R | |
| Aromatic amino acids - F, W, Y | |
| G-glycine | N-asparagine |
| A-alanine | Q-glutamine |
| P-proline | D-aspartic acid |
| V-valine | E-glutamic acid |
| I-isoleucine | K-lysine |
| L-leucine | R-arginine |
| S-serine | F-phenylalanine |
| T-threonine | H-histidine |
| W-tryptophan | Y-tyrosine |
| <E-pyroglutamine | |

Of particular interest in group (b) are the peptides of 8 to 13 amino acids presenting their general formula the carboxy-terminal motif of the oligopeptide:
STRUCTURAL FORMULA [P X¹ X² P P]
where X¹ can be any amino acid and X² is generally I, and the N-terminal amino acid is blocked, generally by <E.

The peptides described in the above table show higher selectivity for the C-site of the ACE as characterized by the Ki values, ranging from 2 nM to 100 μM, while for the N site of the ACE these values were above 50 μM. These peptides are also inhibitors of NEP with Ki values ranging from 5 to 150 μM. All of them potentiate the contractile activity of bradykinin on the isolated guinea pig ileum, doubling the contractile effect of bradykinin, in concentrations varying from 5 to 300 nM. The hypotensive effect of bradykinin was also potentiated by 30-80% in concentrations varying from 10-700 nM, increasing the duration of this hypotensive effect from 5 to 15 fold, as compared to bradykinin alone.

The present invention proposes for the first time: an isolation and purification procedure for vasopeptidase peptide inhibitors with anti-hypertensive action, secreted by serpent venom glands, particularly Bothrops jararaca, comprising the following steps:

A—Isolation and Purification of BPPs from the Venom of Bothrops jararaca:

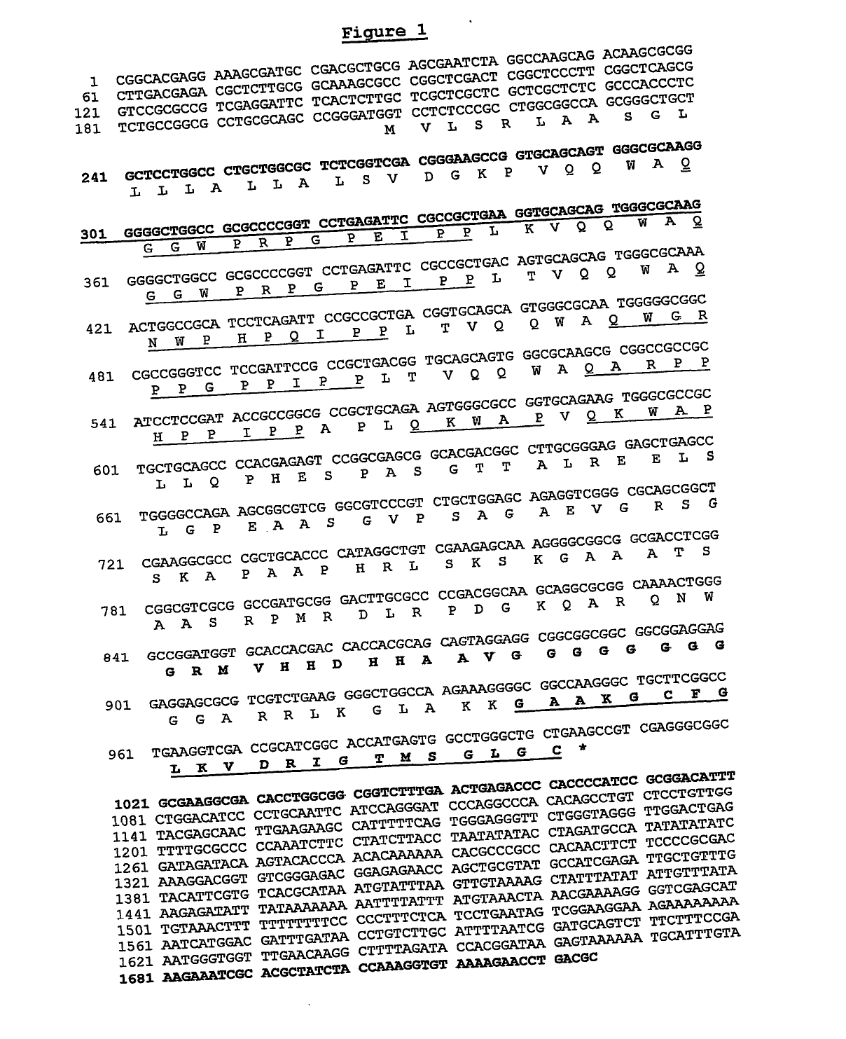

800 to 1500 mg of total venom obtained from a venom pool of Bothrops jararaca were dissolved in 7.0 to 15 ml of deionized water and centrifuged at 1500 to 2000 rpm for 15 to 30 minutes; the supernatant was removed and applied to a 1.2×101 cm Sephadex G-25 M gel filtration column (25-80μ, Sigma); the column was equilibrated with ammonium acetate buffer (30 to 50 mM, pH 5.0 to 6.0) at room temperature. The sample was added to the top of the column and the components were eluted at a flow rate of 1.0 to 2.0 mL/min. The absorbance profile for each aliquot at 214 nm defined the constitution of the pools, based on the bradykinin potentiating activity on guinea pig ileum.
   B—Partial Isolation of the Pool Components Showing Bradykinin Potentiating Activity:
High performance liquid chromatography—HPLC
The components of the pools obtained in step A, showing bradykinin potentiating activity, were partially isolated by high performance liquid chromatography—HPLC (Merck-Hitachi model L-6200A), with the UV-vis detector set at 214 nm, and the reverse-phase column C-18/Beckman (5μ, 4.6×250 mm). Solvent A was 0.5 to 1% TFA (trifluoracetic acid) in H₂O, and solvent B was a 5% to 60% gradient of acetonitrile (ACN), at a flow rate of 0.2 to 0.5 mL/min. The gradients used varied with the sample. The peptides were obtained by manual collection of the absorbance peaks at 214 nm.
   C—Determination of the Molecular Mass and of the Primary Structure of the Bradykinin Potentiating Peptides by Mass Spectrometry (ES-MS-MS).
Another aspect of the invention is related to the procedure for the amino acid sequence determination of the BPPs purified from serpent venom, more specifically Bothrops jararaca, by mass spectrometry performed in the Micromass Quattro II mass spectrometer (ESMS-MS/Micromass), in the positive ionization mode, with an electrospray ion source (Micromass), and the Mass Lynx software (Micromass) used for data acquisition. The samples were dissolved in 50% H₂O/ACN with 0.1% formic acid, and injected with a constant flow of 5 μL/min, by means of an injection pump. The data were acquired in the first quadrupole (ESMS), by scanning the ratio mass/charge (m/z) between 400 and 1600 with a scanning time of 5 seconds during the whole analysis process. Sequencing was performed on selected peptide, displaying a protonated ion, as characterized in the first quadrupole, followed by fragmentation by induced dissociation collision (IDC) with a pressure of argonium of 3×10⁻³, and finally, the data were acquired by scanning at the second quadrupole (ESMS-MS). The software Mass Lynx (Micromass) was used for data acquisition. The machine's parameters were optimized using synthetic bradykinin as a standard. Characterization and sequencing can also be performed with the HPLC system coupled to the mass spectrometry system (LC-ESMS-MS), using the Hewlett-Packard HPLC model 1100 with an automatic injector, the UV-vis detector set at 214 nm, and the reverse-phase column C-18/The Separations Group (4.6×250 mm/5μ), for the isolation of total venom components Solvents used were: Solvent A: 0.1% TFA/H₂O-Solvent B: 10% A/ACN solvent, with a flow rate of 0.6 mL/min. The gradient used was:
t=0-5 min: 0% B
t=5-65 min: 0-60% B
t=65-70 min: 60-100% B
t=70-75 min: 100% B
t=75-80 min: 100-0% B
   Out of the 600 μL/min flow used in the HPLC, 20 μl were automatically injected into the mass spectrometer, and the remaining 580 μl went to the UV-vis detector.
   Mass spectrometry was accomplished in a Micromass Quattro II (ESMS—MS/Micromass) mass spectrometer in the positive ionization mode with an ion electrospray source (Micromass). The acquisition of the characterization and sequencing data was performed by means of a software created to this end, the Mass Lynx software (Micromass).
Another aspect of this invention is the determination of the amino acid sequence of the BPPs by deduction of the cDNA sequence of the precursors of these molecules expressed in serpent tissues, specifically Bothrops jararaca, through the following steps:
Total RNA from the brain of a single B. jararaca specimen was isolated by the guanidine isothiocyanate-phenol-chloroform extraction method, and the messenger RNA was purified by passing the total RNA solution twice through a pre-packed oligo-dT cellulose column. The integrity of the purified mRNA was checked by agarose gel electrophoresis followed by hybridization with radioactive probes, composed of sequences encoding the precursor of the BPPs and the CNP, previously identified by Northern blot assays. Preparation of the cDNA library in λ ZAP phages (Stratagene) was carried out using 5 μg of this sample, following the manufacturer's instructions by common and usual methods. The initial packaging of the recombinant phages generated a titer of approximately 2×10⁵ pfu/mL, with less than 1% of non-recombinant clones This library was immediately amplified, with a final titer of approximately 9×10⁹ pfu/mL.
   F—PCR Amplification (Polymerase Chain Reaction):
The goal of the initial approach was to amplify the cDNA of interest from B. jararaca brain cDNA libraries by PCR, using oligonucleotides specific for the cDNA sequence encoding the precursor of the BPPs and the CNP. The reactions were carried out with materials and methods described here and it was possible to clone only one fragment of approximately 250 base pairs from the total phage lysate from the serpent brain cDNA library (there was no amplification of any other fragment from the serpent brain library). Complete sequencing of this cDNA insert showed a very high degree of similarity with the cDNA sequence identified from the venom gland library, except for the insertion of a few nucleotides (12 bases) in the segment that precedes the natriuretic peptide and some point mutations (4 residues substituted), of which three are silent (encoding the same amino acid residue) and the fourth was “conservative” (a substitution for an amino acid with the same chemical characteristics). In spite of the numerous amplification attempts, changing reaction conditions, and/or the polymerase and oligonucleotide used, it was not possible to clone other fragments from any of the libraries studied.
Approximately 5×10⁶ clones were independently analyzed using the segment encoding the natriuretic peptide as a template for the synthesis of radioactive probes used for the hybridization experiments, which made it possible to identify only 13 positive clones. The plasmid vectors containing the cDNA inserts were recovered by mini-preparations of DNA and then sequenced using oligonucleotides, which anneal to sequences adjacent to the plasmid vector's (pBluescript SK+) multiple cloning site (commercial primers T3 and T7).
Clone selection by hybridization of the cDNA library from the brain of B. jararaca allowed the isolation of a cDNA encoding the precursor for the BPPs and the CNP. (FIG. 1). By analogy to the BPPs isolated from the venom of Bothrops jararaca, 7 peptides were identified, whose sequences are listed among those in table 1). Based on these sequences, peptides were synthesized that presented the same pharmacological properties as the venom peptides, as well as inhibition of ECA and NEP.

FIG. 1 shows the primary sequence of the EVASINS precursor from Bothrops jararaca brain, deduced from the corresponding cDNA sequence. Abbreviations for the nucleotides: A—adenosine; C—cytidine; G—guanosine; T—thymidine. CNP—Type C natriuretic peptide. Nucleotide sequence of the cDNA encoding the precursor protein of the EVASINS and CNP, isolated from the cDNA library from Bothrops jararaca brain, and the deduced amino acid sequence. The amino acid sequences of the EVASINS are underlined and the CNP sequence is shown in bold.

Another aspect of the invention is the procedure for the synthesis of the peptides in solid phase, using two specific strategies:

A—Use of the Tert-Butyloxycarbonyl Group (Boc) as a Temporary Protector for the Amino Group and of Benzyl Derivatives (Bzl) for the Protection of most of the Amino Acid Reactive Side Chains.

The first step in this synthesis strategy is to remove the Boc group from the first amino acid residue, bound to the resin.

Removal of the Boc group occurs in 30% TFA in dichloromethane (DCM), containing 2% anisole, for 30 min. The resin is then washed successively with isopropanol containing 0.5% anisole, DCM and MeOH. For the next amino acid coupling, the amino group is deprotonated by treatment with TEA 10% or DIPEA 5% in DCM for 10 min. The peptidyl-resin is then washed with DCM, MeOH, DCM and with the solvent used for the coupling.

Coupling of the amino acid starts with an activation phase, usually accomplished with coupling agents: diisopropylcarbodiimide (DIC) or 2-(1H-benzotriazolyl)-1,1,3,3-tetramethyllurone tetrafluorborate (TBTU). Usually the Boc-amino acid (carboxylic component, CC) and the coupling agent are used in a 3-fold molar excess, as compared to the amount of amino groups (amine component, CA) in the resin.
When DIC is used, the proportion of the reagents CA:CC:DIC is 1:3:3. When TBTU is used, the proportion of the reagents CA:CC:TBTU:DIEA is 1:3:3:4. Final concentration of these reagents is between 0.05 and 0.1 M.
When coupling Asn (asparagine) or Gln (glutamine), hydroxibenzotriazol (HOBt) is used with the coupling agent to prevent formation of nitrite from these amino acids. For these couplings the final proportion of the reagents CA:CC:TBTU:HOBt:DIEA is 1:3:3:3:5. The coupling reaction, which normally takes 2 hours, is monitored using the ninidrine qualitative method, in which a blue staining of the peptidyl-resin is indicative of incomplete coupling. In this case, recoupling is generally performed changing the solvent used in the previous coupling reaction and/or the coupling agent itself. When the coupling phase is finished, the next cycle is initiated with the deprotection of the amino group until the next residue is coupled. The process continues in cycles until the end of the elongation of the desired sequence.

At the end of the synthesis procedure, the peptide is cleaved from the resin, and its side chains are deprotected in a single step by treatment with anhydrous HF at 0° C. for 60-90 min in the presence of p-cresol and dimethylsulfide (DMS) (5%, v/v of each) which act as suppressors for collateral reactions induced by carbocations released during the HF treatment. When the peptide sequences contain Trp residues, ethanol-1,2-dithiol (EDT) is added to remove the formyl group that protects the side chain of this amino acid simultaneously to the cleavage. EDT is used in the same proportion as the other suppressors (5% v/v, of each). After cleavage, the resin is washed with ethyl acetate and the peptide is eluted by washing the resin with 5% (v/v) acetic acid in water, and lyophilized. A white powder is obtained, generally amorphous.

B—Use of the Base-Labile Protector 9-fluorenylmetoxicarbonile (Fmoc) and Tert-Butyl Derivatives (tBu).

Differently from the previous strategy, in this case the amino group protector Fmoc is used, and removed from the amino acid by treatment with a 20% piperidine solution in DMF for about 20 min. This treatment deprotonates the amino groups for the forthcoming coupling step The coupling methods are the same as the Boc chemistry's and the final cleavage is performed using a weaker acid solution, because both the protector's bond to the side chains (generally of the tert-butylic type) and the peptide's bond to the resin are more labile than in the Boc chemistry's. Usually, the resin is treated with 85-95% TFA (v/v) and a mixture of different suppressors of collateral reactions for about 2 h. After this treatment, the peptide is precipitated with the resin in ice-cold ethylic ether, and centrifuged at a speed of 2,500 g for 5 min. The supernatant is discarded and the pellet is resuspended in ethylic ether, vortexed and centrifuged. The procedure is repeated 5 times and the resin/peptide mixture is dried under vacuum. Finally, the peptide is removed from the resin with 5% AcOH in water (v/v) and filtered. The filtered solution is lyophilized until an amorphous white powder is obtained.

Both strategies A and B are based on the following references: 1—Barany, G. & Merrifield, R. B. (Gross, E. & Meinhofer, J., Eds.) (1980), The Peptides: Analysis, Synthesis and Biology, vol. II, 1, Academic Press, New York. 2—Stewart, J. M. & Young, J. D. (1984), Solid Phase Peptide Synthesis, Pierce Chemical Company, Rockford. 3—Fields, G. B. (1997), Methods in Enzymology, Academic Press, California; 3—Atherton, E. & Sheppard, R. C. (1989), Solid Phase Peptide Synthesis: A Practical Approach, I.L.R. Press, Oxford. 4-Fields, G. B. & Noble, R. L. (1990) Int. J. Pep. Prot. Res. 35, 161.

Following the methods described in this invention, a pool of isolated peptides is obtained, presenting bradykinin potentiating activity, as tested by its contractile activity on smooth muscle, caused by bradykinin, and measured using a preparation of isolated guinea pig ileum, as shown in Example 5.

To determine the bradykinin potentiating activity, a log-dose response curve of the effect of bradykinin on the isolated guinea pig ileum was plotted.

The hypotensive effect of the bradykinin potentiating activity was tested in normotensive and hypertensive (SHR) male and female Wistar rats, anesthetized with ethylic ether.

In the present invention, the peptides of particular interest are natural anti-hypertensive peptides. These will eventually be used as such, or in association with other substances. These synthetic molecules can be conjugated to a variety of ligands, such as acid groups, like sulfonyl, carboxyl and phosphoryl, or other groups, such as thiols, olefins, dithio, azo or diazo compounds, aldehydes and similar compounds.

BPPs bound to specific ligands (peptide aptamers), can be used to identify target proteins, such as enzymes and receptors. For example, specific cells can present target proteins that may be recognized or affected by these modified BPPs. Thus, the use of specific ligands conjugated to the BPPs can drive these molecules preferentially to the cells that possess the specific target proteins. These ligands can be, for example, methylene blue, crystal violet, aspirin, saccharin or others.

In addition to these, several other compounds can be used as ligands, such as steroids, low density lipoproteins, growth factors, viral proteins, etc. Compounds containing such ligands may be preferentially identified by immunoglobulins or their fragments, recognizing them as antigens, wherever they are located. The immunoglobulins of interest are some of the subtypes of IgA, IgD, IgM, IgE and IgG. Immunoglobulins can be derived from any source, particularly from mice and humans The immunoglobulins can be derived from hybridomas, such as transformed lymphocytes, or obtained by recombinant DNA methods.

Particularly, the variable part of the mice immunoglobulin can be linked to the human immunoglobulin, to form a chimeric immunoglobulin with low immunogenicity. Only the use of the Fab fragments, F(ab′)2, Fv, or similar, would be necessary.

Ligands can be coupled to the toxins in question, in order to help them enter the cells and identify the target protein(s) inside. Disulfide bonds can be used to release the toxins from the ligands as they are reduced inside the cell.

The compounds in question can be used in vivo and in vitro. For the in vivo use the compounds can be administered trans-mucosa, parenterally, or by injection, particularly intravenously. The dosage will possibly vary from around 1 μg to 10 mg, usually 0.5 mg/kg of body weight.

Depending on the means of administration and the synthetic toxin used, presenting the same amino acid sequence as the naturally occurring toxin, or a modified form, the compound may eventually be used at a very low dosage. The compound may eventually be diluted in a physiologically acceptable medium, such as a phosphate-saline buffer, saline solution or other more convenient solution.

These compounds may eventually be used in cases of chronic or acute cardiovascular disturbances, in which the micro-circulation would be the target of the treatment. In particular, these compounds may eventually be used in human hypertensive disturbances and their consequences, such as those causing vascular lesions, hyperplasia, etc. A possible recommended use might be in cases of acute pulmonary edema. They may eventually be used in modified or unmodified form, for pathophysiological diagnosis, where the identification or interaction of these toxins with the target proteins (enzymes, receptors, etc.) can be useful.

The BPPs and EVASINS in this invention are used in cardiovascular pathologies.

The innovations presented in this invention, in general terms, consist in adding to the less than 20 known BPPs, dozens of other synthetic peptides with amino acid sequences identical to the natural BPPs and EVASINS, or their chemically modified homologues, which present structural motifs defined here, as well as in characterizing their biological actions through their inhibitory activity on the carboxylic site of the angiotensin converting enzyme (ACE) and also on NEP and ECE

The peptides with these characteristics will be generically designated as BPPs (bradykinin potentiating peptides) and EVASINS (endogenous vasopeptidase inhibitors). The BPPs and EVASINS from this invention correspond to the peptides secreted by the venom gland and to peptides produced in endogenous tissues of Bothrops jararaca.

The sequences of the BPPs were determined by mass spectrometry; the sequences of the EVASINS were determined by deduction of the nucleotide sequence of the cDNA encoding the precursors of these molecules, expressed in serpents, specifically Bothrops jaraxaca.

### EXAMPLES

### Example 1

Partial Isolation of the Pool Components Showing Bradykinin Potentiating Activity:

High Performance Liquid Chromatography—HPLC

The components of the peptide pools displaying bradykinin potentiating activity were partially isolated by high performance liquid chromatography—HPLC Merck-Hitachi model L-6200A, UV-vis detector set at 214 nm, with a reverse phase column: C-18/Beckman (4.6×250 mm/5μ resin) in 0.5% TFA (trifluoracetic acid) in H₂O, and a 5% to 60% gradient of acetonitrile (ACN) at a flow rate of 0.2 to 0.5 mL/min. The gradients used varied according to the sample. The peptides were obtained by manually fractionating the eluate according to the shape of the absorbance peaks at 214 nm.

Purification of the Anti-Hypertensive Peptides from the Venom of the Brazilian Serpent Bothrops jararaca.

The total venom, obtained from a venom pool of Bothrops jararaca (1200 mg) supplied by the Venom Section of Instituto Butantan, was dissolved in 10 mL of deionized water and centrifuged at 800 g for 30 min. The supernatant was collected and loaded onto a Sephadex G-25 (25-80μ, Sigma) gel filtration column (1.2×101 cm). The column was equilibrated with 30 mM ammonium acetate buffer, pH 5.5 at room temperature. The sample was loaded onto the top of the column and the components were eluted with a flow rate of 1.2 mL/min. The absorbance profile of each aliquot, at 214 nm, defined the constitution of the pools.

High Performance Liquid Chromatography—HPLC

The HPLC equipment used was a Merck-Hitachi, model L-6200A UV vis detector set at 214 nm, with a reverse phase column: C-18/Beckman (4.6×250 mm/5μ resin) for the partial isolation of the pools with bradykinin potentiating activity, in 0.5% TFA (trifluoracetic acid), and a 5% to 60% gradient of acetonitrile (ACN) at a flow rate of 0.5 mL/min.

### Example 2

Determination of the Molecular Mass and of the Primary Structure of the Bradykinin Potentiating Peptides by Mass Spectrometry

The Mass spectrometry experiments were performed in a Micromass Quattro II mass spectrometer (ESMS-MS/Micromass) in the positive ionization mode with an electrospray ion source (Micromass). The Mass Lynx software was used for data acquisition.

The mass spectrometer's parameters were optimized using synthetic bradykinin as a standard.

The samples were dissolved in 50% ACN/H₂O and 0.1% formic acid, and injected at a constant flow rate of 0.5 μL/min, by means of an injection pump. The data were acquired at the first quadrupole (ESMS), by scanning of the mass/charge ratio (m/z) between 400 and 1600 using a scanning time of 5 seconds during the whole analysis procedure.

The peptide, with protonated ion as characterized at the first quadrupole, was sequenced and fragmented by collision of induced dissociation (CID) with a pressure of argonium gas of 3×10⁻³, and, finally, the data were acquired by scanning at the second quadrupole (ESMS-MS).

HPLC System Coupled to the Mass Spectrometer (LC-ESMS-MS).

The HPLC system used was a Hewlett-Packard model 1100, with automatic injector, UV-vis detector set at 214 nm, with a reverse phase column: C-18/Beckman (4.6×250 mm/5μ resin) for the isolation of components from total venom.
Solvent A: 0.1% TFA/H₂O
Solvent B: 10% solvent A/ACN, with a flow rate of 0.6 mL/min.
   The gradient used was:
t=0-5 min: 0% B
t=5-65 min: 0%-60% B
t=65-70 min: 60%-100% B
t=70-75 min: 100% B
t=75-80 min: 100%-0% B
   The HPLC flow rate was 600 μL/min, out of which 20 μl were automatically injected into the mass spectrometer, while the remaining 580 μl were sent to the UV-vis detector.
   Mass spectrometry was carried out in a Micromass Quattro mass spectrometer (Micromass). Sequencing and characterization data were acquired using a software specially developed for this end.

### Example 3

Synthesis of Peptides in Solid Phase

Two strategies were used for peptide synthesis.

The first, more traditional strategy, uses the tert-butyloxicarbonyl group (Boc) as a temporary protector for the amino group and benzyl derivatives (Bzl), for the protection of most of the reactive side chains of the amino acids.

The second and most recent strategy alternates between the base labile protector 9-fluorenylmetoxicarbonyl (Fmoc) and tert-butyl derivatives (tBu).

a) The Boc/Bzl strategy—The first step in this strategy is the removal of the Boc group from the first amino acid residue bound to the resin.

Removal of the Boc group occurs in 30% TFA in dichloromethane (DCM), containing 2% anisole, for 30 min. The resin is washed with isopropanol containing 0.5% anisole, DCM and MeOH. For the coupling of the forthcoming amino acid, the amino group is deprotonated (neutralized) with 10% TEA or 5% DIPEA in DCM for 10 min. The peptidyl-resin is then washed with DCM, MeOH, DCM and the solvent to be used in the coupling phase.

Coupling of the amino acid starts with an activation phase, usually accomplished with coupling agents: diisopropylcarbodiimide (DIC) or 2-(1H-benzotriazolyl)-1,1,3,3-tetramethyllurone tetrafluorborate (TBTU). Usually the Boc-amino acid (carboxylic component, CC) and the coupling agent are used in a 3-fold molar excess, as compared to the amount of amino groups (amine component, CA) in the resin.

When DIC is used, the proportion of the reagents CA:CC:DIC is 1:3:3. When TBTU is used, the proportion of the reagents CA:CC:TBTU:DIEA is 1:3:3:4.

Final concentration of these reagents is between 0.05 and 0.1 M. When coupling Asn (asparagine) or Gln (glutamine), hydroxibenzotriazol (HOBt) is used with the coupling agent, to prevent the formation of nitrile from these amino acids.

For these couplings the final proportion of the reagents CA:CC:TBTU:HOBt:DIEA is 1:3:3:3:5. The coupling reaction, which normally takes 2 hours, is monitored using the ninidrine qualitative method and a blue staining of the peptidyl-resin is indicative of incomplete coupling. In this case, recoupling is generally performed changing the solvent used for the previous coupling and/or the coupling agent.

When the coupling phase is finished, the next cycle is initiated with the deprotection of the amino group until the next residue is coupled. The process goes on cyclically until the end of the elongation of the desired sequence.
At the end of the synthesis procedure, the peptide is cleaved from the resin and its side chains are deprotected in a single step by treatment with anhydrous HF at 0° C. for 60-90 min in the presence of p-cresol and dimethylsulfide (DMS) (5%, v/v each) which act as suppressors for collateral reactions induced by carbocations released during the HF treatment.

When the peptide sequences contain Trp residues, ethanol-1,2-dithiol (EDT) is added to remove the formyl group that protects the side chain of this amino acid simultaneously with the cleavage. EDT is used at the same proportion as the other suppressors (5% v/v, each). After cleavage, the resin is washed with ethyl acetate and the peptide is eluted by washing the resin with 5% (v/v) acetic acid in water, and lyophilized. A white powder is obtained, generally amorphous.

b) The Fmoc/tBu Strategy

Differently from the previous strategy, the amino group protector Fmoc is used, which is removed from the amino acid by treatment with a 20% piperidine solution in DMF for about 20 min. This treatment deprotonates the amino groups for the next coupling step. The coupling methods are the same as the Boc chemistry's and the final cleavage is performed using a weaker acid solution because both the protector's bond to the side chains (generally of the tert-butylic type) and the peptide's bond to the resin are more labile then the Boc chemistry's. Usually, the resin is treated with 85-95% TFA (v/v) and a mixture of different suppressors of collateral reactions for about 2 h. After this treatment, the peptide is precipitated with the resin in ice-cold ethylic ether, and centrifuged at 2,500 g for 5 min. The supernatant is discarded and the pellet is resuspended in ethylic ether, vortexed and centrifuged. The procedure is repeated 5 times and the resin and peptide mixture is dried under vacuum. In the end, the peptide is released from the resin with 5% AcOH in water (v/v) and filtered. The filtered solution is lyophilized until an amorphous white powder is obtained.
1—Atherton, E. & Sheppard, R. C. (1989), Solid Phase Peptide Synthesis: A Practical Approach, I.L.R. Press, Oxford.
2—Fields, G. B. & Noble, R. L. (1990) Int. J. Pep. Prot. Res. 35, 161.

### Example 4

Peptide Sequences Deduced from the cDNA Encoding the Precursors of the BPPs.

Northern Blot Assays

Total RNA from several tissues of Bothrops jararaca was isolated using the extraction method with guanidine isothiocianate-phenol-chloroform. Ten micrograms of total RNA from each of the serpent's tissues were separated by denaturing agarose gel electrophoresis (1.7% formaldehyde), and transferred by capillarity to nylon membranes. The RNA was blotted to the membrane at 80° C. In a vacuum-oven for 1 hour followed by UV treatment in a Crosslinker (model RPN 2500—Amersham), at 70.000 μJ/cm² for 5 minutes.

The membranes were pre-hybridized at 42° C., over night (approximately 16 hours), in a buffer containing 50% formamide, 2.5 mM K₂PO₄, 5× Denhardt's solution, 50 μg/mL herring sperm DNA and 10% dextran sulfate.

The cDNA fragments used as templates for the synthesis of radioactive probes were obtained by digestion of the clone containing the cDNA insert encoding the BPPs and CNP (clone NM87, Murayama et al., Proc. Natl. Acad. Sci. USA, 94, 1189-1193, 1997), with the appropriate restriction enzymes (Sma I or Sma I+Bam HI, for the sequences encoding the BPPs and the CNP, respectively).

The excised inserts were separated by low melting agarose gel electrophoresis and the DNA was recovered by phenol/chloroform extraction followed by ethanol precipitation.

The radioactive probes were prepared using approximately 25 ng of template DNA, obtained as described above, for each reaction, following the instructions of the manufacturer of the RediPrime kit (Amersham), based on the incorporation of α[³²P]-dCTP in the presence of random hexanucleotides and polymerase (Klenow or T4 DNA polymerase). The non-incorporated radioactive nucleotides were separated from the probe in inverse molecular sieve columns (“spin columns S-200” Pharmacia).

Hybridizations with the radioactive probes were carried out at 42° C., for 16 hours, after adding the radioactive probe to the pre-hybridization solution at a concentration of approximately 1.5×10⁶ cpm/mL.

Finally, the membranes were washed at high stringency conditions, i.e., washed four times at 65° C. with a 2×SSC; 0.1% SDS solution for 15 minutes each, and three times at 65° C. In a 0.1×SSC; 0.1% SDS solution. The membranes were exposed to X-rays films (LS-Kodak) in appropriate cassettes, for the appropriate time, depending on the strength of the radioactive signal observed for each membrane. For a better detection of the weaker signals, the cassettes, contained a pair of screens, the film and the membrane, and were kept at −80° C.

Purification of the Messenger RNA and Preparation of the cDNA Library from B. jararaca Brain

Total RNA from the brain of B. jararaca was isolated using the guanidine isothiocyanate-phenol-chloroform extraction method.

The messenger RNA was purified by passing the total RNA solution twice through a pre-packed oligo-dT cellulose column (GibcoBRL). One aliquot of the purified messenger RNA was submitted to denaturing agarose gel electrophoresis (containing 1.7% formaldehyde) followed by staining with ethidium bromide. The RNA was transferred by capillarity to nylon membranes to confirm the integrity of the samples through hybridizations with probes specific for the sequences encoding the BPPs. The preparation of the cDNA library in λ ZAP phages was carried out using a cDNA preparation kit from Stratagene (La Jolla, Calif.) After cloning the double stranded cDNA inserts, obtained from 5 μg of brain messenger RNA into the λ ZAP phages, these were packaged in vitro and titered to verify the cloning efficiency. This phage library was amplified and the aliquots were stored at −20° C. and −80° C., in the presence of chloroform and DMSO, respectively.

Amplification of the cDNA from the Total Phage Lysate

Using 10 μl of the total phage lysate obtained for the brain cDNA library, PCR (Polymerase Chain Reaction) amplification was carried out using specific oligonucleotides, derived from the sequence of the cDNA encoding the precursor of the BPPs and the CNP from the venom gland. The phage lysate was initially incubated at 100° C. for 5 minutes, then cooled down to 4° C. and, finally, added to the other components of the amplification reaction, composed of 10 pmol of each primer, 2.5 U of Taq polymerase (Amersham), 200 μM dNTPs and 1×PCR buffer, supplied with the enzyme. The reactions thus prepared were then submitted to a denaturing cycle at 94° C. for 4° C. and, then, to 35 more cycles of 1 min at 94° C., 1 min at 60° C., 1 min at 72° C. Subsequently, the reactions were kept at 4° C. until the PCR products were analyzed by agarose gel electrophoresis, after staining with ethidium bromide. The PCR products of the expected size, based on the cDNA sequence from the venom gland, were subcloned in a plasmid vector pCRscript SK+ (Stratagene), following the manufacturer's instructions (by usual methods), and sequenced using oligonucleotides that anneals to adjacent regions of the vector's multiple cloning site (primers T3 and T7).

Selection and Identification of the Clone Encoding the Precursor of the BPPs:

The fragments obtained by digestion of clone NM87 with Sma I or Sma I and Bam HI (approximately 450 bp and 432 bp, positions 164-610 and 610-1044, respectively) were used as templates for the synthesis of radioactive probes (α³²P) using the random primer method (Rediprime kit/Amersham). The nitrocellulose membranes (Schleicher & Schuell) prepared from plates containing approximately 50 thousand phage plaques each, were submitted to hybridization with the radioactive probes in 6×SSPE (1×SSPE: 0.15 M NaCl, 15 mM NaH₂PO₄, pH 7, 1 mM EDTA), 50% formamide, 0.1% SDS and 5× Denhardt's at 42° C. for 16 hours. The membranes were then washed twice in 2×SSC/0.1% SDS and three times in 0.1×SSC/0.1% SDS, at 65° C., for 15 minutes each (1×SSC: 0.15 M NaCl, 15 mM sodium citrate, pH 7).

Positive phage plaques were identified by autoradiography and were isolated for the analysis of the DNA insert In vivo excision of the phagemid pBluescript from the vector λ ZAP was carried out using the “helper phage”, following the instructions of the manufacturer, and the extremities of the insert were sequenced.

Sequencing of the DNA Inserts:

The sequencing reactions were carried out following the chain termination method with dideoxy-nucleotides, using the kit Big Dye (Perkin Elmer), followed by the analysis in the ABI 310 automatic sequencer, following the manufacturer's instructions.

Total RNA from the brain of a single B. jararaca specimen was isolated by the guanidine isothiocyanate-phenol-chloroform extraction method, and the messenger RNA was purified by passing the total RNA solution once through a pre-packed oligo-dT cellulose column. The integrity of the purified mRNA of the sample was checked by agarose gel electrophoresis followed by hybridization with radioactive samples synthesized from de cDNA encoding the precursor of the BPPs and the CNP (identified by Northern blot assays).

Preparation of the cDNA library in λ ZAP phages (Stratagene) was carried out by common and usual methods using 5 μg of this sample, following the manufacturer's instructions.

The initial packaging of the recombinant phages obtained generated a titer of approximately 2×10⁵ pfu/mL, with less than 1% of non-recombinant clones. This library was immediately amplified, with a final titer of approximately 9×10⁹ pfu/mL.

PCR (Polymerase Chain Reaction) Amplification

The goal of the initial approach used was to amplify the cDNA of interest from B. jararaca brain cDNA library by PCR, using oligonucleotides specific for the cDNA sequence encoding the precursor of the BPPs and the CNP. The reactions were carried out with materials and methods described herein. Only one fragment of approximately 250 base pairs was cloned from the total phage lysate containing the brain cDNA library (there was no amplification of any other fragment from the serpent brain or pancreas library). Complete sequencing of this cDNA insert showed a very high degree of similarity with the cDNA sequence identified in the venom gland library, except for the insertion of a few nucleotides (12 bases) in the segment that precedes the natriuretic peptide and some point mutations (4 residues substituted), of which three are silent (encoding the same amino acid residue) and the fourth was “conservative” (a substitution for an amino acid with the same chemical characteristics). In spite of the numerous amplification attempts changing reaction conditions and/or the polymerase and oligonucleotide used, it was not possible to clone other fragments from any of the libraries studied.

Approximately 5×10⁶ clones were independently analyzed using the segment encoding the natriuretic peptide as a template for the synthesis of radioactive probes used for the hybridization experiments, which made it possible to identify 13 positive clones. The plasmid vectors containing the cDNA inserts were recovered by mini-preparations of DNA and sequenced using oligonucleotides that anneal to adjacent regions of the plasmid vector's (pBluescript SK+) multiple cloning site (commercial primers T3 and T7).

Screening of clones by hybridization of the cDNA library from the brain of B. jararaca allowed the isolation of a cDNA encoding the BPPs as part of a precursor protein for these peptides (FIG. 1). Seven peptides (the sequences of which are listed among those in table 1). were identified by analogy with the BPPs isolated from the venom of Bothrops jararaca. Based on these sequences, peptides were synthesized that presented the same pharmacological properties as the venom peptides, as well as inhibition of ECA and NEP.

### Example 5

Testing the Bradykinin Potentiating Activity of the BPPs and EVASINS:

Biological Assay on Guinea Pig Ileum:

The potentiating activity of the pools of isolated peptides was tested on the contractile activity of bradykinin on smooth muscle, measured using a preparation of isolated guinea pig ileum. Female guinea pigs with a body weight between 160 and 180 g were used. Before starting the assays, the ileum was kept in TYRODE solution at 37° C. for 30 min. One of the ends of the isolated ileum segment, measuring 1.5 to 2.0 cm, was tied to a semi-ring located at the bottom of a glass container with a capacity of 5 mL, containing the TYRODE physiological solution at 37° C. under constant oxygen bubbling through a capillary. The other extremity of the ileum segment was tied to a previously calibrated registering arm. The tension on the arm was of 1 g, and the ileum contractions were recorded by an REC101 recorder (Pharmacia Biotech).

The samples were prepared in deionized water at the time of use, and the volume used for the biological preparation did not exceed 0.2 mL.

In order to determine the bradykinin potentiating effect of the samples, a log-dose curve of the effect of bradykinin on the ileum was plotted. Bradykinin activity was determined by measuring the contractions of the isolated guinea pig ileum, and the potentiating activity was expressed as a function of the increase in tissue response to a standard dose of bradykinin.

Pools and fractions were assayed individually and were added 30 seconds before the addition of a simple dose of bradykinin. The measured response was interpolated in the linear section of the log dose-effect curve, giving the potentiating activity as a function of the raise in tissue response to a standard dose of bradykinin. TYRODE solution: 20 mL of stock solution I, 40 mL of the solution II, 1 mL of difenidramine solution (1 mg/mL), 1 mL of atropine solution (1 mg/mL), 5.60 mM D-glucose and H₂O to 1 L.

All reagents used in this experiment were of analytic grade.

### Example 6

Effect on the Systemic Blood Circulation and Microcirculation

The hypotensive potentiating activity effect of bradykinin was tested in normotensive and hypertensive male and female Wistar rats, anesthetized with ethylic ether; a cannulae was introduced in the femoral vein for drug administration and in the femoral artery for blood pressure recording. A Gould polygraph was used coupled to a Statham Gould physiological transducer. The arterial pressure variation values were obtained by integration of the areas delimited by the pressure base line, and by comparing them with the values obtained in the control experiment. Toxin effect tests were carried out in normotensive and hypertensive rats by intra-vital microscopy using the mesenteric circulation. The animals were anesthetized, and their body temperature maintained by a heated plate, while the mesentery was trans-illuminated and observed through lenses coupled to a television camera. The vase diameters of these animals were measured with a micrometric screw. Tested substances were administered by a constant flow peristaltic pump or injected in bolus.

### Example 7

Enzymatic Assays for the Determination of ACE Inhibition by the BPPs.

The wild-type human angiotensin converting enzyme (ACE) and two mutants, containing only one functional active site, were obtained through stable transfection of chinese hamster ovary cells with the ACE encoding cDNA. The two ACE mutants were expressed as full-length proteins, presenting one of the catalytic sites, N- or C-terminal, inactivated by substitution of the zinc binding histidine residues by lysine residues.

The enzymatic assays were carried out at 25° C. with the substrate Mca-Ala-Ser-Asp-Lys-DpaOH, in a 50 mM Hepes buffer, pH 6.8, containing 200 mM NaCl and 10 μM ZnCl₂. The reactions were continuously monitored by determining the raise in fluorescence at 390 nm emission wave length, and excitation at 340 nm, caused by the cleavage of the substrate (S=Km, 40 μM) by ACE, in a fluorimeter Fluorolite 10000 (Dynatech). The bradykinin potentiating peptides (BPPs) (inhibitors, were pre-incubated with the enzyme 90 min before the addition of the substrate.

### Example 8

Characterization of the Effect of the BPPs In Vivo-Arterial Blood Pressure and Microcirculation

Wistar male rats with a body weight between 180 and 220 g were anesthetized with sodic pentabarbital (Hypnol® Cristalia, 50 mg/kg of body weight, intraperitoneally), while the body temperature was maintained between 36.5° C. and 37° C. by a heated plate. After tracheotomy, the ventilation of the animals was controlled by a mechanical ventilator (Harvard Rodent Ventilator, model 683, Harvard Apparatus, South Natick, Mass., USA), under standard conditions, as follows: frequency: 57-65 inspirations/min; passing volume: 2-2.5 mL; inspired oxygen fraction: 0.25-0.40. Cannulae were introduced in the right carotid artery and the jugular vein (PE-50 catheters, 58 mm ID, Portex, Hythe, UK) for the continuous monitoring of the arterial blood pressure (TSE System Technical & CHM Scientific Equipment GMBH), and for the administration of liquids, respectively. The mesenteric bed was exposed. After trichotomy of the abdominal region, the skin was ruptured and the mesentery was reached through an incision at the alba line. The mesentery was exposed under controlled temperature on the transparent area of the plate, which was placed on the microscope charriot. The temperature of the plate was kept at 37° C. The exposed tissue was kept moist and heated by perfusion with Ringer-Locke solution, pH 7.2-7.4 containing 1% gelatin (154 mM NaCl; 5.6 mM KCl; 2 mM CaCl₂.2H₂O; 6 mM NaHCO₃; 5 mM glucose) at 37° C.

The study of mesenteric microcirculation in situ was carried out using an optical microscope (Akioskop-Carl Zeiss, Germany) coupled to a colored image projection camera (TVC-TKC 6000), which transmits, simultaneously or not, the images obtained in the microscope to a computer and/or to a TV monitor. Image analysis software, MS Windows compatible, (Kontron KS 300, Kontron Bild Analyse-GMBH, Germany), is installed in the computer allowing quantitative measurements of the stored images at fixed time intervals. A video tape recorder capable of recording the entire course of the experiment is connected to the TV monitor.

Thus, the images visualized during the experiment can be quantitatively evaluated through the fixed image on the computer's monitor, and at the same time the course of the experiment observed on the TV monitor can be stored by the video tape recorder for later analysis.

The optical microscope has a system of amplifying lenses (Optovar), localized between the lenses and the projection camera. The image transmitted to the monitors results from magnifications determined by the lenses, amplifying lenses and projection camera. In the studies described here, magnification on the computer monitor was of 860 times and on the TV monitor, of 1530 times.

Determination of the red blood cells speed in the microcirculatory vessels (diameters between 15 and 30 μm) was carried out by the photometric method described by BORDERS & GRANGER (1984) and complemented by Davis (1987). Through an optical doppler velocimeter (Microcirculation Research Institute, Texas A&M College of Medicine), installed between the eye piece of the optical microscope and the image projection camera of the intravital microscopy equipment, the blood flow was determined in situ on the computer monitor. The equipment is composed of two sensors (Planar photodiodes and low capacitance), which are placed at the center of the image of the vessel under observation on the computer monitor. The sensors, at an established and known distance between them, are capable of detecting alterations in the light intensity produced by the passage of red blood cells and transform them into voltage signals, which are proportional to the speed of those cells.

The sensors determine the diameter of the vessel and the speed of the red blood cells at the center of the blood flow.

Experimental Protocols

Surgical Procedures
10 minutes for stabilization of the microcirculatory net and blood pressure.
Administration of BPPs or equivalent volume of sterile saline solution (5 mL in 10 minutes). Immediately after administration of BPPs or saline solution, injection of bradykinin in bolus (3 μg/300 μl).

1. A process for the isolation and purification of vasopeptidase peptide inhibitors, showing specificity for the carboxyl site of the angiotensin-converting enzyme, secreted by serpent venom glands (BPPs), particularly BOTHROPS JARARACA, or produced endogenously (EVASINS), having vasodilating and anti-hypertensive action, comprising of the following steps:
a) providing venom, obtained from a pool of B. jararaca venom dissolved in deionized water followed by centrifugation at 1500-2000 rpm for 15 to 20 minutes thereby producing a supernatant; passing the supernatant through a gel-filtration column, equilibrated with 30-50 mM ammonium-acetate buffer, pH 5.0 to pH 6.0, at room temperature;
b) conveying the sample to the top of the column and then eluting the components at a flow rate of 1.0 to 2.0 mL/min;
c) obtaining the absorbance profile for each aliquot at 214 nm in order to define the constitution of the pools
d) partially purifying the components of the pools showing potentiating activity by high performance liquid chromatography, for which 0.1 to 1.0% TFA (trifluoro acid)/H₂O and acetonitrile/H₂O (9:1) are used as solvents, and a gradient of 5% to 60% of solvent B, with a flow rate of 0.2 to 0.5 mL/min
e) determining the molecular mass and the primary structure of the bradykinin potentiating peptides by mass spectrometry (ESMS-MS).

2. The process according to claim 1, characterized by the fact that in step (d) an HPLC Merck-Hitachi, model L-6200A is used, with the UV-vis detector set at 214 nm, and the reverse-phase column C-18/Beckman (5μ, 4.6×250 mm).

3-22. (canceled)

23. A vasopeptidases inhibitor with anti-hipertensive and vasodilating action, characterized by having a formula selected from the group consisting of:

| | |
|---|---|
| I | pp¹aa¹aa²aa³P⁴aa⁵aa⁶P⁷P⁸, |
| II | pp¹aa¹aa²aa³aa⁴P⁵aa⁶aa⁷P⁸P⁹, |
| III | pp¹aa¹aa²aa³aa⁴aa⁵P⁶aa⁷aa⁸P⁹P¹⁰, |
| IV | pp¹aa¹aa²aa³aa⁴aa⁵aa⁶P⁷aa⁸aa⁹P¹⁰P¹¹, |
| V | pp¹aa¹aa²aa³aa⁴aa⁵aa⁶aa⁷P⁸aa⁹aa¹⁰P¹¹P¹², |
| and | |
| VI | pp¹aa¹aa²aa³aa⁴aa⁵aa⁶aa⁷aa⁸P⁹aa¹⁰aa¹¹P¹²P¹³, |

where:
P is always proline and the remaining amino acids are always L-amino acids and are presented in the following one-letter code;
pp¹ is the N-terminus;
aa¹ is a non-basic amino acid; aa² is a non-acid amino acid;
aa³ is a non-acid amino acid;
aa⁴ is an amino acid;
aa⁵ is an amino acid;
aa⁶ for formula II, aa⁷ for formula III, aa⁸ for formula IV, and aa⁹ for formula V is a non-basic amino acid;
aa⁶ for formula I, aa⁷ for formula II, aa⁸ for formula III, aa⁹ for formula IV, and aa¹⁰ for formula V are always I or A or T wherein:

| | |
|---|---|
| G-glycine | N-asparagine |
| A-alanine | Q-glutamine |
| P-proline | D-aspartic acid |
| V-valine | E-glutamic acid |
| I-isoleucine | K-lysine |
| L-leucine | R-arginine |
| S-serine | F-phenylalanine |
| T-threonin | H-histidine |
| W-tryptophane | Y-tyrosine |
| <E-pyroglutamyl | |

24. The vasopeptidase inhibitor with vasodilation and anti-hypertensive action, according to claim 23, characterized by the fact that D and E are acidic amino acids, K and R are basic amino acids, and F, W, and Y are aromatic amino acids.

25. The vasopeptidase inhibitor with vasodilation and anti-hypertensive action, according to claim 23, characterized by the fact that they are peptides of 8-13 amino acids presenting a general formula, which contains the sequence motif at the carboxyl-terminus of the oligopeptide:
P X¹ X² P P
where X¹ can be any amino acid and X² usually is I, and the N-terminal amino acid is blocked, and P is proline.

26-33. (canceled)

34. The vasopeptidase inhibitors of claim 23 diluted in a physiogogically acceptable carrier.

35. (canceled)

36. A process for using the vasopeptidase inhibitor of claim 23 in vivo, via trans-mucosa, parenterally, or by injection, and/or intravenously for systemic action or localized action in tissue microcirculation.

37. A process for using the vasopeptidase inhibitor of claim 23 in vivo, in dosages varying between approximately 1 g to 10 mg/kg of body weight.

38. A peptide of Formula I:
pp¹aa¹aa²aa³P⁴aa⁵aa⁶P⁷P⁸ (I)
wherein:
(a) pp¹ is the N-terminus of the peptide;
(b) aa¹ is a non-basic amino acid;
(c) aa² is a non-acid amino acid;
(d) aa³ is a non-acid amino acid;
(e) P⁴ is proline;
(f) aa⁵ is an amino acid selected from the group consisting of glutamine, asparagine, proline, and glycine;
(g) aa⁶ is an amino acid selected form the group consisting of isoleucine, alanine, and threonin;
(h) P⁷ and P⁸ are proline.

39. A peptide of Formula II:
pp¹aa¹aa²aa³aa⁴P⁵aa⁶aa⁷P⁸P⁹ (II)
wherein:
(a) pp¹ is the N-terminus of the peptide;
(b) aa¹ is a non-basic amino acid;
(c) aa² is a non-acid amino acid;
(d) aa³ is a non-acid amino acid;
(e) aa⁴ is an amino acid;
(f) P⁵ is proline;
(g) aa⁶ is a non-acid amino acid;
(h) aa⁷ I, is an amino acid selected form the group consisting of isoleucine, alanine, and threonin;
(i) P⁸ and P⁹ are proline.

40. A peptide of Formula III:
pp¹aa¹aa²aa³aa⁴aa⁵P⁶aa⁷aa⁸P⁹P¹⁰ (III)
wherein:
(a) pp¹ is the N-terminus of the peptide;
(b) aa¹ is a non-basic amino acid;
(c) aa² is a non-acid amino acid;
(d) aa³ is a non-acid amino acid;
(e) aa⁴ is an amino acid;
(f) aa⁵ is an amino acid selected from the group consisting of glutamine, asparagine, proline, and glycine;
(g) P⁶ is proline;
(h) aa⁷ is a non-acid amino acid;
(i) aa⁸ I, is an amino acid selected form the group consisting of isoleucine, alanine, and threonin;
(j) P⁹ and P¹⁰ are proline.

41. A peptide of Formula IV:
pp¹aa¹aa²aa³aa⁴aa⁵aa⁶P⁷aa⁸aa⁹P¹⁰P¹¹ (IV)
wherein:
(a) pp¹ is the N-terminus of the peptide;
(b) aa¹ is a non-basic amino acid;
(c) aa² is a non-acid amino acid;
(d) aa³ is a non-acid amino acid;
(e) aa⁴ is an amino acid;
(f) aa⁵ is an amino acid selected from the group consisting of glutamine, asparagine, proline, and glycine;
(g) aa⁶ is a non basic amino acid;
(h) P⁷ is proline;
(i) aa⁸ is a non-acid amino acid;
(j) aa⁹ I, is an amino acid selected form the group consisting of isoleucine, alanine, and threonin;
(k) P¹⁰ and P¹¹ are proline.

42. A peptide of Formula V:
pp¹aa¹aa²aa³aa⁴aa⁵aa⁶aa⁷P⁸aa⁹aa¹⁰P¹¹P¹² (V)
wherein:
(a) pp¹ is the N-terminus of the peptide;
(b) aa¹ is a non-basic amino acid;
(c) aa² is a non-acid amino acid;
(d) aa³ is a non-acid amino acid;
(e) aa⁴ is an amino acid;
(f) aa⁵ is an amino acid selected from the group consisting of glutamine, asparagine, proline, and glycine;
(g) aa⁶ is an amino acid;
(h) aa⁷ is a non basic amino acid;
(i) P⁸ is proline;
(j) aa⁹ a non-acid amino acid;
(k) aa¹⁰ I, is an amino acid selected form the group consisting of isoleucine, alanine, and threonin;
(l) P¹¹ and P¹² are proline.

43. A peptide of Formula VI:
pp¹aa¹aa²aa³aa⁴aa⁵aa⁶aa⁷aa⁸P⁹aa¹⁰aa¹¹P¹²P¹³ (VI)
wherein:
(a) pp¹ is the N-terminus of the peptide;
(b) aa¹ is a non-basic amino acid;
(c) aa² is a non-acid amino acid;
(d) aa³ is a non-acid amino acid;
(e) aa⁴ is an amino acid;
(f) aa⁵ is an amino acid selected from the group consisting of glutamine, asparagine, proline, and glycine;
(g) aa⁶ is an amino acid;
(h) aa⁷ is an amino acid;
(i) aa⁸ is a non basic amino acid;
(j) P⁹ is proline;
(k) aa¹⁰ is an amino acid selected from the group consisting of glutamine, asparagine, proline, and glycine;
(l) aa¹¹ I, is an amino acid selected form the group consisting of isoleucine, alanine, and threonin;
(m) P¹² and P¹³ are proline.

44. The peptide of any one of claims 38 through 43 wherein pp¹ is pyroglutamyl.

45. The peptide of any one of claims 38 through 43 wherein aa¹ is an amino acid selected from the group consisting of tryptophane, serine, glycine and asparagine.

46. The peptide of any one of claims 38 through 43 wherein aa² is an amino acid selected from the group consisting of proline, glycine, tryptophane, and arginine.

47. The peptide of any one of claims 38 through 43 wherein aa³ is an amino acid selected from the group consisting of proline, alanine, arginine, and tryptophane.

48. The peptide of any one of claims 38 through 43 wherein aa⁴ is an amino acid selected from the group consisting of threonin, proline, glycine, histidine, arginine, tryptophane, and glutamic acid.
